(19) United States
(12) Patent Application Publication
(10) Pub. No.: US 2006/0241971 A1
(43) Pub. Date: Oct. 26, 2006

(54) My SmileGuide

(76) Inventor: Fred Shakespeare Ferguson, East Setauket, NY (US)

Correspondence Address: FRED S. FERGUSON, 10 BRAEMER ROAD, EAST SETAUKET, NY, 11733, US

(21) Appl. No.: 11/113,167

(22) Filed: Apr. 25, 2005

(51) Int. Cl. G06Q 10/00; A61B 5/00

(52) U.S. Cl. 705/2; 600/300

(57) ABSTRACT

An oral health risk assessment and educational tool, which provides demographic, longitudinal and cross sectional health, and attitude data and management of these data sets for clients and users.

### RELATED APPLICATIONS

Oral health information is available on the internet and through printed materials from health providers, clinics, special interest groups and through professional schools. Various insurance companies and oral health product manufacturers, not for profit organizations and special interest groups provide oral health content to members and the general public through computers (the internet). Current systems are primarily “read only” or provide general (non-specific) responses. If there are risk assessment programs that also provide the kind of response presented in the invention and software management of data, it is probably limited in health programs of the insurance carrier provided through to their “members only” web site. We are aware of no such program that would provide the kind of assessment or the level of counseling of the invention.

### FEDERALLY SPONSORED RESEARCH OR DEVELOPMENT

None

### SEQUENCE LISTING

Not applicable

### BACKGROUND OF THE INVENTION

The present invention relates to the methods, processes and content of information presented to an entity for the purpose of providing health assessment and risk evaluation based on information collected from the entity that is directly and indirectly related to oral health and other health characteristics.

An entity can be a single individual, a group of individuals, a corporation, an institution or any community sharing a common interest.

The information presented to the entity depends on several factors such as demographics or behavioral and health information provided by the entity.

The information is captured and presented to the entity through computer software made available on the internet by the invention, or through computer software made available on a CD-ROM by the invention.

The invention provides a much improved method of providing information and managing data related to the entity requesting oral health counseling and to those interested in the resultant data for various reasons.

The invention was developed by Dr Fred Ferguson as a mean to provide individualized reliable oral health counseling and develop a business strategy of marketing his vision of oral health knowledge, behavioral health, developing strategic partnerships and providing valuable data.

The first program developed by Dr Ferguson is a web site launched in 1999, www.AboutSmiles.org, which is a read-only presentation. AboutSmiles.org averages 9,000 unique monthly visitors.

Dr Ferguson is a professor of pediatric dentistry of the School of Dental Medicine, Stony Brook University with a long history of professional student education and development of health programs for pediatrics and special needs.

### BRIEF SUMMARY OF THE INVENTION

The invention is based on information, processes and methods, which present a questionnaire program to users and caregivers. The purpose of the questionnaire is to determine risk behaviors well recognized to be related to common oral health problems. In addition the interactive tool provided by the questionnaire, the invention also presents the user with tools designed to enhance the user's oral health care and knowledge.

Each questionnaire and each tool is tailored to each user based on age group and gender on the one hand, and health history on the other hand.

Upon completing the questionnaire, the user receives a report containing predetermined replies that address their risk concerns and provides counseling with the intent to change the behavior of the user (or caregiver) and with the purpose of reducing such user's (or caregiver's) risk.

The report contains replies corresponding to each of the question in the questionnaire, provided that a risk behavior has been identified. Each reply provides the user with information explaining the reason why a specific answer represents a risk and also provides counseling on how a user should alter their behavior to reduce such risk.

The invention utilizes computer software to manage the data processing (e.g. user demographics, risk questionnaire and market replies). The invention also provides reports to the user specific to various health providers, which in turn will help the user (self or caregiver) provide appropriate oral health activity (self) or supervision (as a caregiver of a child) and “helper” subjects specific to situations determined by age or certain situations that are associated with oral health. All information captured by the invention is stored in a centralized database.

These products are saved or printed from the user's computer. Specifically: Reports for health providers:
Oral health report for the pediatric health provider: Specific to American Academy and Centers for Disease Control policy for pediatric health providers “to do an oral health risk assessment for children by 12 months of age”.
Check list for dental visits: The user will enter the results of the dental visit to the invention and will in turn receive counseling related to problems identified in the check list.
Oral health report for dental visits: Provides the provider the results of the user risk profile

Tools printouts (examples)
Snack contract: Provides the caregiver or individual a means for behavior modification.
Health tips for braces: Provides the individual health counseling to reduce the risk for problems associated with orthodontic appliances.

In addition to immediate risk assessment and counseling, the invention also provides other benefits for individuals, communities and corporations:
Collective statistical information
   The invention can provide statistical feedback for each questionnaire (e.g.: how many individuals chose a specific answer to a specific question) as well as statistical information based on selectable demographic criteria (e.g.: risk chart for caregivers of 1 year old children allowing to establish an average score for a specific population)
Individual or Collective progress information
   Because each individual can take the same questionnaire several times, it is possible to measure the success of each reply and modify or clarify each reply accordingly. This information is available individually (e.g.: an individual can find out whether s/he is making progress in his/her oral care) or collectively (e.g.: A graph showing the percentage of individuals who are showing progress).

In general, since all information is captured in a centralized database, all data mining processes, methodologies and technologies already available in the market place become accessible and allow for the preparation and presentation of simple or complex reports with regards to all statistical information gathered through the invention.

As the invention continues to be developed, additional risk assessments programs, reports and response surveys will be added. Through the progress information reports, existing risk assessments programs will be revised to better transmit the risk information to each individual.

The invention has currently been developed primarily for use on the Internet. It can also be presented as a CD-ROM program or in printed form depending on the level of technology of the user community (e.g. case managers trained as “oral health educators” can also provide the questionnaire one on one).

1. The purpose of the invention is to provide a means for business development by licensing its use to “clients” and presentation to society with a means for corporate sponsorship. Each client can have their own internet “folder” so as to maintain their own data or direct their members to our server. Clients include:
Insurance and HMO companies: Health education for members and employees.
School districts: Health education curricula for students, staff and teachers.
Employers and unions health and wellness programs for employees.
Early child-care companies (e.g. Kindercare): caregivers of enrolled children.
Any client who would want to have their “own” folder of the invention for the exclusive use of their members or consumers
Placement of a corporate (e.g. product manufacturer) banner on the publicly available web site, www.MySmileGuide.com. The publicly available web site may at some point present a fee for the general public.
The software program provides the client with data specific to health behavior and disease outcomes (insurance company), marketing and sales potential (product vendor), and user response data.
The data (user or member demographics) itself would be a product for different corporate product manufacturers. We will be developing an array of questionnaires specific to user opinion and health behaviors as the invention is developed.
